(19) United States
(12) Patent Application Publication
(10) Pub. No.: US 2005/0101844 A1
(43) Pub. Date: May 12, 2005

(54) System and method for linking patient monitoring data to patient identification

(76) Inventors: David Wayne Duckert, Menomonee Falls, WI (US); Donald Eugene Brodnick, Cedarburg, WI (US); James Steven Cincotta, New Berlin, WI (US)

Correspondence Address: MICHAEL BEST & FRIEDRICH LLP, 100 EAST WISCONSIN AVENUE, MILWAUKEE, WI, 53202-4108, US

(21) Appl. No.: 10/704,429

(22) Filed: Nov. 7, 2003

(51) Int. Cl. A61B 5/00; G06F 17/60

(52) U.S. Cl. 600/300; 235/375; 705/2

(57) ABSTRACT

A patient monitoring system and method for associating acquired clinical data with a patient. The system can include a patient identification device that can be coupled to a patient, a data acquisition device that can be coupled to the patient to acquire clinical data, a patient monitor coupled to the medical acquisition device, a monitor identification device coupled to the patient monitor, and a code reader that can be used by a caregiver to read the patient identification device and the monitor identification device.

### BACKGROUND OF THE INVENTION

In the near future it is expected that bar code readers will be pervasive in the healthcare industry, and particularly at the point-of-care (i.e., the physical location at which the patient receives treatments and/or medications). In an effort to reduce medical errors, the Food and Drug Administration recently proposed new regulations concerning the bar coding of medications. Ideally, the patient, the medication, and the caregiver would each bear a bar code. A bar code reader at the point-of-care enables verification of the proper dosage of medication, verification of the correct patient, verification that the medication will not have adverse effects due to the patient's allergies or due to interactions with other medications, and finally, verification that the caregiver is authorized to administer the medication. Such systems are already being used in some hospitals, such as the Veteran's Administration Hospitals. Many hospitals are currently choosing wireless bar code readers because of the convenience of transportation and avoidance of tangled cords.

Similar to the concept of linking a patient and the patient's medication is the concept of linking a patient and the patient's clinical data at the point of care in a bedside patient monitor. Conventionally, in order to link a patient and the patient's clinical data, the patient's name must be either (a) entered via a tedious character-by-character process, often resulting in errors or mismatched records (e.g., Mike vs. Michael) or (b) requested from the hospital information system based on the bed number and unit name (e.g., bed number 16 in the ICU) for the physical location of the particular patient monitor. This second method can be problematic in that the hospital information system often contains old data (e.g., the patient may have moved and the hospital information system was not updated) or the patient is not in the assigned bed (e.g., the patient is in an operating room, an x-ray room, etc.).

Legal and efficiency pressures are forcing hospitals to maintain patient data in an electronic Clinical Data Repository (CDR). It is imperative that this data be marked with the correct patient identification. In the case of a patient monitor being physically connected to a bar code reader, the association between the patient identification and the patient's clinical data generated by the patient monitor is explicit. The patient identification is linked to the patient monitor via the physical connection to the bar code reader. In other words, when the caregiver uses a bar code reader that is hardwired to the patient monitor, the patient identification data read by the bar code reader is automatically received by the patient monitor and automatically saved with the patient's monitoring data. However, with a wireless bar code reader, the patient identification data read by the bar code reader is not automatically received by the patient monitor and saved with the patient's monitoring data. Rather, at least one additional step must be taken for the patient monitor to receive the patient identification and save it with the patient's monitoring data. For example, a hospital ward may have five patients and ten beds with patient monitors, and the patients may be continually moved into and out of the hospital ward. When a new patient arrives, he or she is placed in an open bed. A wireless bar code reader can be used to capture the patient's identification, but currently there is no adequate method to determine which one of the ten patient monitors is displaying data for this patient. It may be possible for a patient monitor to generate a visual or audible signal that alerts the caregiver that the patient monitor emitting the signal is generating data for the particular patient being scanned with the bar code reader. However, this method can be ineffective if another caregiver in the same room is scanning a different patient at the same time. Similar problems can occur with wireless patient monitors or with patient monitors that do not have an adequate user interface for use by the caregiver to enter the patient's name (such as conventional sub-acute products).

Furthermore, some patient monitors are portable and do not have networking capabilities (wired or wireless). The caregivers must use a manual process to transfer the patient's clinical data (including, but not limited to, heart rate, blood pressure, blood oxygen level, etc.) from these non-networked patient monitors to the patient's electronic record in the hospital information system. This manual data transfer process is often error prone and time consuming.

### BRIEF DESCRIPTION OF THE INVENTION

In light of the above, a reliable system for linking a patient's identification with the patient's clinical data generated from various patient monitors in a hospital and/or a clinic would be desirable.

One embodiment of the invention provides a patient monitoring system including a patient identification device that can be coupled to a patient, a data acquisition device that can be coupled to the patient to acquire clinical data, a patient monitor coupled to the data acquisition device, a monitor identification device coupled to the patient monitor, and a code reader that can be used by a caregiver to read the patient identification device and the monitor identification device so that the acquired clinical data is associated with the patient.

Some embodiments of the invention provide a patient monitoring system including a data acquisition device that can be coupled to a patient to acquire clinical data, a patient monitor coupled to the medical acquisition device, a clinical data code displayed on the patient monitor, and a code reader. The clinical data code can represent at least a portion of the acquired clinical data, and the code reader can be used by a caregiver to read the clinical data code in order to obtain at least a portion of the acquired clinical data from the patient monitor.

### BRIEF DESCRIPTION OF THE DRAWINGS

FIG. 1 is a schematic illustration of a patient monitoring system according to one embodiment of the invention.

FIG. 2 is a schematic illustration of a patient monitoring system according to another embodiment of the invention.

FIG. 3 illustrates one embodiment of a bar code for encoding patient clinical data.

### DETAILED DESCRIPTION OF THE INVENTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limited. The use of “including,” “comprising” or “having” and variations thereof herein is meant to encompass the items listed thereafter and equivalents thereof as well as additional items. The terms “mounted,” “connected” and “coupled” are used broadly and encompass both direct and indirect mounting, connecting and coupling. Further, “connected” and “coupled” are not restricted to physical or mechanical connections or couplings, and can include electrical connections or couplings, whether direct or indirect.

In addition, it should be understood that embodiments of the invention include both hardware and electronic components or modules that, for purposes of discussion, may be illustrated and described as if the majority of the components were implemented solely in hardware. However, one of ordinary skill in the art, and based on a reading of this detailed description, would recognize that, in at least one embodiment, the electronic based aspects of the invention may be implemented in software. As such, it should be noted that a plurality of hardware and software based devices, as well as a plurality of different structural components may be utilized to implement the invention. Furthermore, and as described in subsequent paragraphs, the specific mechanical configurations illustrated in the drawings are intended to exemplify embodiments of the invention and that other alternative mechanical configurations are possible.

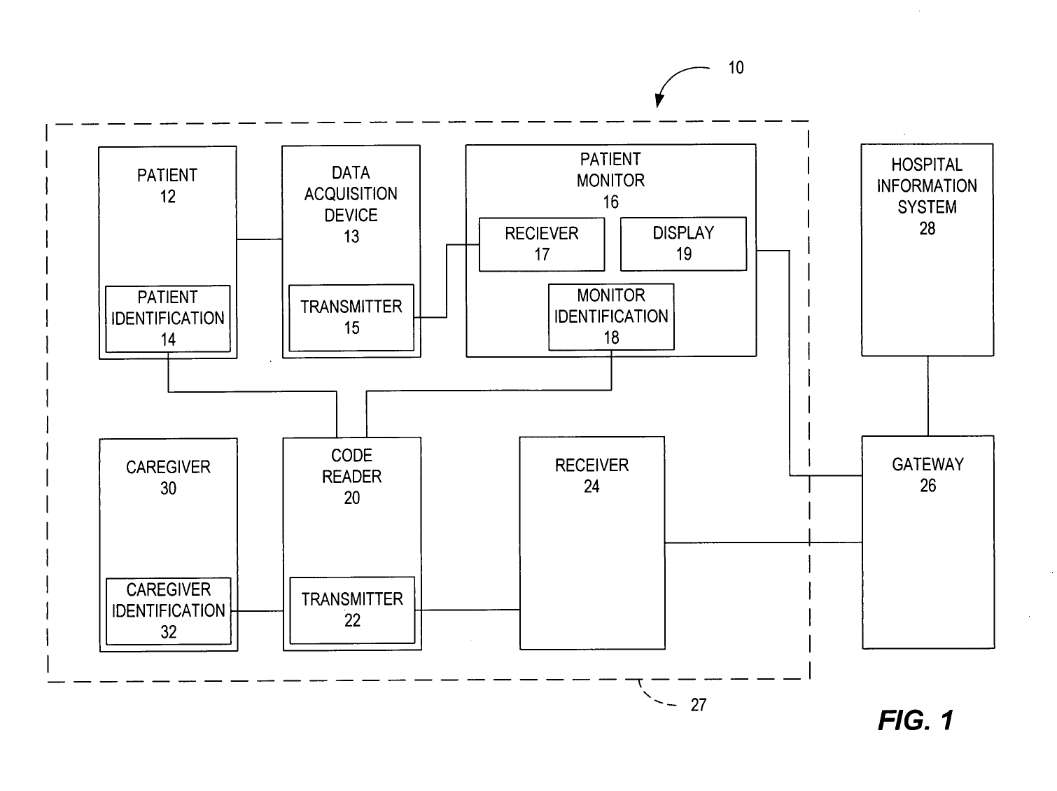

FIG. 1 illustrates a patient monitoring system 10 according to one embodiment of the invention. The patient monitoring system 10 can be used to associate at least two of the following: a patient identification, a monitor identification, a caregiver identification, and patient clinical data (i.e., any data acquired from the patient that has any clinical significance). The patient monitoring system 10 can be connected to or associated with a patient 12 having a patient identification 14. The patient monitoring system 10 can include a data acquisition device 13 for acquiring clinical data from the patient 12, a patient monitor 16 (having a monitor identification 18) for displaying the acquired clinical data, a code reader 20 (that includes a transmitter 22), a receiver 24, a gateway 26, and a hospital information system 28 (which allows patient clinical data to be stored and retrieved electronically). Although not required, the patient monitoring system 10 can be connected to or associated with a caregiver 30 having a caregiver identification 32.

Clinical data can be obtained from the patient 12 using a variety of data acquisition devices or equipment commonly known to those of ordinary skill in the art. The clinical data can be displayed on the patient monitor 16 in any suitable manner. For example, the clinical data can include, without limitation, at least one of heart rate, blood oxygen saturation, blood pressure, respiration rate, body temperature, electrocardiogram (ECG), and any other clinical data that may be required depending on the patient's condition and the care being provided. A variety of corresponding data acquisition devices 13 can be used to obtain the patient's clinical data, such as a heart rate monitor, a pulse oximetry sensor, a manual or automatic sphygmomanometer, a thermometer, electrocardiology equipment, and any other data acquisition device 13 capable of obtaining clinical data from the patient 12. In some embodiments, the data acquisition device 13 can be a part of the patient monitor 12. In other embodiments, the data acquisition device 13 can be independent of the patient monitor 12 and connected to the patient monitor 12 via a hardwired or wireless connection, as described below.

Referring to FIG. 1, clinical data obtained from the patient 12 can be transmitted, via hardwired or wireless connections, to the patient monitor 16 and therefrom to the gateway 26. All connections are represented by a solid line, but it should be understood that solid lines can represent hardwired or wireless connections. The gateway 26 serves as a connection between a Life Critical Network 27 within a hospital or other healthcare-providing entity and the hospital information system 28. The Life Critical Network 27 can be an internal network within the healthcare-providing system that is used, for example, for all patient alarms and data acquisition hardware and/or software within the healthcare-providing entity. The Life Critical Network 27 can generally only be accessed and manipulated by authorized personnel, and is typically a more sensitive and protected network environment than the hospital information system. The gateway 26 in some embodiments of the invention can translate the clinical data into a form that can be used by the hospital information system 28. The gateway 26 can also serve to protect critical patient data within the Life Critical Network 27 from any outside source or from any unauthorized manipulation.

The hospital information system 28 can be a central information repository for an entire healthcare-providing system (i.e., a Clinical Data Repository). That is, the hospital information system 28 can contain patient information that can be accessed by a diverse population of administrative and medical staff members within the healthcare-providing system. The hospital information system 28 can be accessed from a laptop, a desktop, or any other workstation in any department or area within the healthcare-providing system including, without limitation, a pharmacy, a billing department, an accounting department, a nurses' station, an operating room, an emergency room, an intensive care unit, a cathlab, an x-ray room, or any other department or sector within the healthcare-providing system. As a result, the hospital information system 28 is available to a much broader range of healthcare personnel and can be a slightly more open network than the Life Critical Network 27 guarded by the gateway 26.

Clinical data obtained from the patient 12 and displayed on the patient monitor 16 can also be transmitted to the hospital information system 28 via the hardwired or wireless connection between the patient monitor 16, the gateway 26, and the hospital information system 28. The data acquisition device 13 can include a transmitter 15 and the patient monitor 16 can include a receiver 17 for receiving a transmitted signal from the transmitter 15. The transmitter 15 can transmit a signal to the receiver 17 via a hardwired or wireless connection.

In some embodiments of the invention, the code reader 20 is hardwired to the patient monitor 16. The code reader 20 can be used to scan the patient identification 14 in order to associate the patient identification 14 with the clinical data that is being displayed on the patient monitor 16 after being acquired by the data acquisition device 13. The patient identification 14 can be transmitted to the hospital information system 28 along with the clinical data being acquired from the patient 12, ensuring that the patient 12 (via the patient identification 14) is associated with the patient's clinical data.

In other embodiments of the invention, the code reader 20 is wirelessly connected to the patient monitor 16 and any other component of the patient monitoring system 10. Conventional methods of using wireless code readers can make association of the patient identification 14 and the patient's clinical data difficult and more error-prone. The patient monitoring system 10 of FIG. 1 can include a wireless code reader 20. The wireless code reader 20 can be used to scan or otherwise read the patient identification 14, as well as the monitor identification 18, in order to provide an association or a data link between the patient 12 and the patient monitor 16. The transmitter 22 of the code reader 20 can transmit information relating to the patient identification 14 and the monitor identification 18 to the receiver 24. The receiver 24 can be coupled to the gateway 26 via a hardwired or a wireless connection. In other words, the receiver 24 can be an element of the gateway 26, or can be a separate element that can transmit information to the gateway 26. The patient's clinical data that is associated with the monitor identification 18 can also be associated or linked with the patient's identification 14 and stored together in the hospital information system 28.

In some embodiments of the invention, the patient monitoring system 10 can be associated with a caregiver 30 having a caregiver identification 32 [e.g., the caregiver 30 can have a caregiver identification 32 such as a radio frequency identification (RFID) tag that can send a signal wirelessly to the patient monitoring system 10]. The code reader 20 can be used to scan or otherwise read the caregiver identification 32 and associate the caregiver identification 32 with the patient identification 14 and the monitor identification 18. The caregiver identification 32 can be stored in the hospital information system 28 along with the patient's clinical data in the electronic record for the patient 12. Scanning or otherwise reading the caregiver identification 32 can also serve as an authorization or certification checkpoint or to develop an audit trail of clinical activity at the point-of-care. For example, if a caregiver 30 is not authorized to administer care to a particular patient 12, the patient monitor 16 or another component in the patient monitoring system 10 can alert the appropriate personnel.

The patient identification 14 can be positioned on the patient 12 in an unobtrusive location so that it will not cause discomfort or harm to the patient 12. The patient identification 14 can also be positioned in a location to which it is easily applied (e.g., the patient's wrist or ankle). The patient identification 14 can be physically located on a variety of devices including, without limitation, a bracelet or an ankle strap that cannot be easily removed. The device can be attached to the patient 12 upon being admitted to the healthcare-providing system so that the patient 12 can be associated with his/her patient identification 14 throughout the healthcare-providing process. The caregiver identification 32, if connected to or associated with the patient monitoring system 10, can be located on the caregiver 30 in a location similar to where the patient identification 14 is located on the patient 12, or the caregiver identification 32 can be located on a name tag or a security badge for the comfort and convenience of the caregiver 30.

The monitor identification 18 can be located on the exterior of the patient monitor 16 in any location that is readily accessible to the caregiver using the code reader 20. In some embodiments, the monitor identification 18 can be presented on a device that is coupled to the patient monitor 16 (e.g., a sticker that is adhered to the exterior of the patient monitor 16). In other embodiments, the monitor identification 18 can be integrally formed with a portion of the patient monitor 16. In still other embodiments, the monitor identification 18 can be presented on a display 19 of the patient monitor 16, such as a liquid crystal display (LCD), or any suitable type of computer screen.

As used herein and in the appended claims, any one of the patient identification 14, the caregiver identification 32, or the monitor identification 18 can be referred to as an “identification device.” An identification device can include only the code used for identification, or an identification device can include the code as well as the device to which the code is coupled or on which the code is supported (e.g., wristband, ankle strap, name tag, sticker, display, image, etc.).

In one embodiment, the monitor identification 18 can represent a unit number or a bed number within a hospital or other healthcare-providing system. In another embodiment, the monitor identification 18 can represent an Internet protocol (IP) address assigned to the patient monitor 16. For dynamically-allocated Internet protocol addresses [i.e., when the patient monitor 16 is part of a network that uses a dynamic host configuration protocol (DHCP)], a code representation (e.g., a bar code) for the Internet protocol address can be presented on the display 19 of the patient monitor 16. In some embodiments, code readers that include a charge coupled device (CCD) can be used to read codes that are presented as an image on the display 19 of the patient monitor 16. That is, code readers including a CCD can be used to convert a code presented as an image on the display 19 of the patient monitor 16 into electrical signals which represent the information contained within the code.

In some embodiments, the monitor identification 18 can be presented on the display 19 of the patient monitor 16 without obscuring the patient's clinical data that is also being displayed. For example, the monitor identification 18 can be present on a single frame, or alternating frames, of a video stream being displayed on the patient monitor 16. The monitor identification 18, in some embodiments, can be nearly invisible to a caregiver but readable by the code reader 20.

In other embodiments of the invention, the monitor identification 18 can be presented as a sequenced flicker at one or more points on the display 19 of the patient monitor 16. In such embodiments, the code reader 20 can be shaped and sized similar to a pen or a small probe-like instrument that can be held over the points on the display 19 of the patient monitor 16 where the monitor identification 18 is presented as a sequenced flicker. Monitor identifications 18 that are presented as a sequenced flicker can be serialized in time instead of space. In other words, the flickers can be timed to provide a unique sequence for each patient monitor 16 (as opposed to a bar code presented for the code reader 20 at one time within a defined physical space). By allowing a caregiver to hold the code reader 20 over a location on the display 19 of the patient monitor 16, without requiring that the code reader 20 be moved or swiped over the monitor identification 18, code reading errors can be reduced. That is, the unique sequence presented by the flicker can be automatically presented at a scan speed and scan time that is acceptable by the code reader 20. The use of a sequence flicker generally does not require the caregiver 30 to guess at an appropriate amount of time for holding the code reader 20 over the code, or an appropriate speed at which to swipe the code reader 20 across the code. A variety of suitable codes and bar codes known to those of ordinary skill in the art can be presented as a sequenced flicker on the patient monitor 16. For example, reduced space symbology (RSS) codes (i.e., codes that contain more information than a standard bar code and generally have the appearance of two standard bar codes stacked vertically) can be presented as a sequenced flicker.

In general, the patient identification 14, the caregiver identification 32, or the monitor identification 18 can include a bar code or any other suitable code that represents the identification of the patient 12, the caregiver 30, or the patient monitor 16, respectively, and can be read by any suitable code reader. In some embodiments, the patient identification 14, the caregiver identification 32, or the monitor identification 18 can include a character string of varying lengths and varying characters assigned to the patient 12, the caregiver 30, or the patient monitor 16, respectively. The identification can include, without limitation, a bar code of any symbology or encoding (e.g., a RSS code), a radio frequency identification (RFID) tag (i.e., a tag capable of transmitting a radio frequency signal to a receiver), or other suitable codes or images known to those of ordinary skill in the art, and any suitable combinations thereof. If a RFID tag is used, it can be directional, such that the RFID tag can interact with a particular code reader, but not interact with a different code reader in another area of the room or more than one code reader at once.

According to one embodiment of the method of the invention, the caregiver 30 can use the code reader 20 to scan the patient identification 14 and the monitor identification 18 before, during, or after the caregiver 30 treats, monitors, or administers medication to the patient 12. The caregiver 30 can couple the patient 12 to the data acquisition device 13, acquire clinical data with the data acquisition device 13, and display the clinical data on the patient monitor 16. Once the patient identification 14 and the monitor identification 18 have been scanned, the patient 12 has been associated with the clinical data that is being sent to the hospital information system 28 from the patient monitor 16, and the clinical data being acquired by the data acquisition device 13 can be added to the appropriate patient electronic file.

The patient monitor 16 can be associated with the patient 12 until the caregiver uses the code reader 20 to read a different patient identification, or until the patient 12 is associated with a new patient monitor. The patient 12 can be associated with the patient monitor 16 by following the above-described procedure and using the code reader 20. The clinical data obtained from the patient monitor 16 during this association can be added to the patient's electronic record in the hospital information system 28. The patient 12 can be associated with a new patient monitor in the same or a different location of the hospital or healthcare-providing entity by following the above-described procedure and using the same or a different code reader. The clinical data obtained from the new patient monitor during this new association can be added to the patient's electronic record, and so on.

In addition, if the patient monitor 12 has the capability to display (and/or acquire) more than one patient's clinical data at a time, the patient monitor 12 can have multiple monitor identifications 18, and various data lines or regions of the display 19 of the patient monitor 12 can be associated with different monitor identifications 18. In this regard, the patient 12 can be associated with the patient monitor 16 following the above-described procedure, and a new patient can be associated with the same patient monitor 16 following the above-described procedure and using the same or a different code reader 20. One or more lines or regions of the display 19 can be associated with the patient 12, and one or more different lines or regions of the display 19 can be associated with the new patient. Each patient's electronic record can be populated with the clinical data acquired from that patient.

In some embodiments, the caregiver 30 can use the code reader 20 to read the caregiver identification 30, which can serve as an additional element of information sent to the patient's electronic record and/or can serve to authorize the process of acquiring clinical data from the patient 12. The patient identification 14, the monitor identification 18, and the caregiver identification 32 can be read in any suitable sequence without departing from the spirit and scope of the present invention.

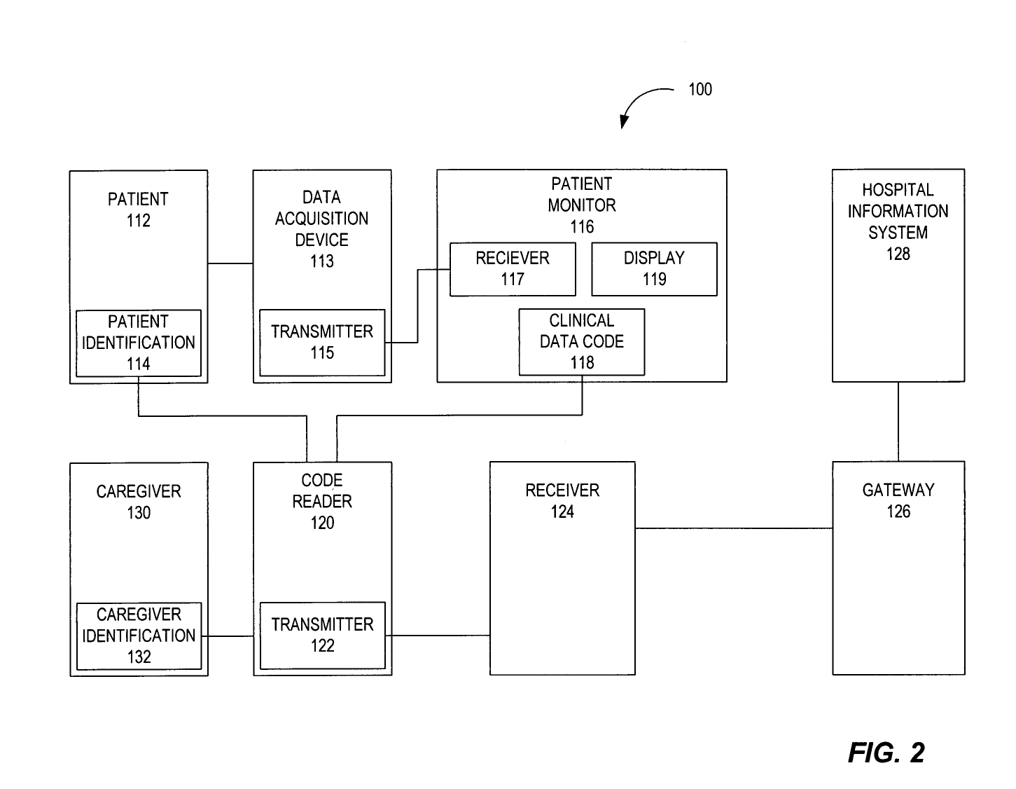

FIG. 2 illustrates a patient monitoring system 100 according to another embodiment of the invention. The patient monitoring system 100 can be connected to or associated with a patient 112 having a patient identification 114. The patient monitoring system 100 can include a data acquisition device 113 (having a transmitter 115) for acquiring clinical data from the patient 112, a patient monitor 116 (having a receiver 117) for displaying clinical data acquired from the data acquisition device 113 on a display 119, a code reader 120 (having a transmitter 122), a receiver 124, a gateway 126, and a hospital information system 128. In some embodiments, the patient monitoring system 100 can be connected to or associated with a caregiver 130 having a caregiver identification 132.

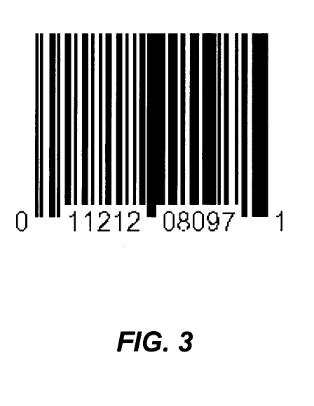

In the embodiment shown in FIG. 2, the patient monitor 116 is not directly connected to the gateway 126. Rather, the code reader 120 is used to transmit the various identifications and the actual clinical data from the patient monitor 116 to the gateway 126 and/or the hospital information system 128. The patient monitor 116 can display patient clinical data that is being acquired using the data acquisition device 113 and can present the acquired clinical data as a clinical data code 118. In other words, rather than representing an identification or in addition to representing an identification, the clinical data code 118 can also represent the actual clinical data that has been acquired from the patient 112. The clinical data code 118 can be presented on the display 119 (such as an LCD or any suitable computer screen) of the patient monitor 116. FIG. 3 illustrates an example of a clinical data code 118. The clinical data code 118 illustrated in FIG. 3 can encode, for example, the following patient vital signs data:
Heart rate: 112 beats per minute;
Blood pressure: 120/80 mmHg; and
Blood oxygen saturation: 97 percent.
   Other information, including non-clinical data, can be encoded in the clinical data code 118 as well. For example, a monitor identification and/or information relating to the monitor type can be encoded in the clinical data code 118.

The code reader 120 can be used to read the patient identification 114, the clinical data code 118, and the caregiver identification 132. The transmitter 122 of the code reader 120 can transmit a signal to the receiver 124 representative of the patient identification 114, the clinical data code 118, and/or the caregiver identification 132. The receiver 124 can transmit the identification information to the gateway 126 and to the hospital information system 128 via various suitable hardwired and/or wireless connections. In this manner, the clinical data acquired from the data acquisition device 113 can be added to the patient's electronic record without requiring any manual recordation of the clinical data.

According to another embodiment of the method of the invention, the caregiver 130 can couple the patient 112 to the data acquisition device 113, acquire patient clinical data with the data acquisition device 113, and display the acquired clinical data on the patient monitor 116. The caregiver 130 can use the code reader 120 to read the patient identification 114 and the clinical data code 118 (which represents the actual clinical data that is being acquired from the patient 112). The caregiver 130 can use the code reader 120 to read the patient identification 114 first and the clinical data code 118 second, or the clinical data code 118 first and the patient identification 114 second. In some embodiments, the caregiver 130 can use the code reader 120 to also read the caregiver identification 132. The caregiver identification 132 can be read at any point in the sequence of reading the patient identification 114, the clinical data code 118, and the caregiver identification 132.

Various features and advantages of the invention are set forth in the following claims.

1. A patient monitoring system comprising:
a patient identification device that can be coupled to a patient;
a data acquisition device that can be coupled to the patient to acquire clinical data;
a patient monitor coupled to the data acquisition device;
a monitor identification device coupled to the patient monitor; and
a code reader that can be used by a caregiver to read the patient identification device and the monitor identification device so that the acquired clinical data is associated with the patient.

2. The patient monitoring system of claim 1 wherein at least one of the patient identification device and the monitor identification device includes a bar code.

3. The patient monitoring system of claim 1 wherein the monitor identification device includes a clinical data code representing at least a portion of the acquired clinical data.

4. The patient monitoring system of claim 1 wherein the monitor identification device and the acquired clinical data are displayed on the patient monitor.

5. The patient monitoring system of claim 4 wherein the monitor identification device does not obscure the displayed clinical data.

6. The patient monitoring system of claim 1 wherein the monitor identification device is coupled to an exterior portion of the patient monitor.

7. The patient monitoring system of claim 1 wherein the monitor identification device includes an Internet protocol address.

8. The patient monitoring system of claim 1 wherein the monitor identification device includes an image displayed on the patient monitor.

9. The patient monitoring system of claim 1 wherein the monitor identification device includes a time-sequenced flicker displayed on the patient monitor.

10. The patient monitoring system of claim 1 wherein the code reader is at least one of a bar code reader connected by a hardwire to the patient monitor and a bar code reader wirelessly connected to the patient monitor.

11. The patient monitoring system of claim 1 and further comprising a caregiver identification device that can be coupled to a caregiver.

12. The patient monitoring system of claim 11 wherein the code reader can read the caregiver identification device in order to associate the caregiver with at least one of the acquired clinical data, the patient, and the patient monitor.

13. The patient monitoring system of claim 11 wherein the code reader can read the caregiver identification device in order to create an audit trail of clinical activity.

14. The patient monitoring system of claim 1 where the data acquisition device includes at least one of a heart rate monitor, a pulse oximetry sensor, a sphygmomanometer, a thermometer, and electrocardiology equipment.

15. The patient monitoring system of claim 1 and further comprising a receiver coupled to the code reader, a gateway coupled to the receiver, and a hospital information system coupled to the gateway.

16. The patient monitoring system of claim 1 wherein the code reader includes a charge coupled device that converts an image into electrical signals.

17. The patient monitoring system of claim 1 wherein the monitor identification device includes an image displayed on the patient monitor in one of a single frame of a video stream and alternating frames of a video stream.

18. The patient monitoring system of claim 1 wherein at least one of the patient identification device and the monitor identification device includes a reduced space symbology code.

19. The patient monitoring system of claim 1 wherein at least one of the patient identification device and the monitor identification device includes a radio frequency identification tag.

20. A method of associating a patient with clinical data acquired from the patient, the method comprising:
reading a patient identification device coupled to a patient;
reading a monitor identification device coupled to a patient monitor;
acquiring clinical data from the patient; and
associating the patient identification device with the monitor identification device in order to associate the acquired clinical data with the patient.

21. The method of claim 20 and further comprising reading a bar code included in at least one of the patient identification device and the monitor identification device.

22. The method of claim 20 and further comprising reading a clinical data code included in the monitor identification device, the clinical data code representing at least a portion of the acquired clinical data.

23. The method of claim 20 and further comprising displaying the monitor identification device and the acquired clinical data on a patient monitor.

24. The method of claim 23 and further comprising displaying the monitor identification device without obscuring the displayed clinical data.

25. The method of claim 20 and further comprising attaching the monitor identification device to an exterior portion of a patient monitor.

26. The method of claim 20 and further comprising displaying a monitor identification device including an Internet protocol address.

27. The method of claim 20 and further comprising displaying a monitor identification device including an image on a patient monitor.

28. The method of claim 20 and further comprising displaying a monitor identification device including a time-sequenced flicker on a patient monitor.

29. The method of claim 20 and further comprising reading at least one of the patient identification device and the monitor identification device with a wireless bar code reader.

30. The method of claim 20 and further comprising reading a caregiver identification device coupled to a caregiver.

31. The method of claim 30 and further comprising reading a caregiver identification device in order to associate the caregiver with at least one of the acquired clinical data, the patient, and a patient monitor.

32. The method of claim 30 and further comprising reading a caregiver identification device in order to create an audit trail of clinical activity.

33. The method of claim 20 and further comprising acquiring clinical data including at least one of heart rate, blood oxygen saturation, blood pressure, respiration rate, body temperature, and electrocardiogram data.

34. The method of claim 20 and further comprising transmitting the acquired clinical data from a code reader to a receiver from the receiver, to a gateway, and from the gateway to a hospital information system.

35. The method of claim 20 and further comprising displaying a monitor identification device including an image, reading the image with a code reader having a charge coupled device, and converting the image into electrical signals.

36. The method of claim 20 and further comprising displaying the monitor identification device with one of a single frame of a video stream and alternating frames of a video stream.

37. The method of claim 20 and further comprising displaying a monitor identification device including a reduced space symbology code.

38. The method of claim 20 and further comprising displaying a monitor identification device including a radio frequency identification tag.

39. A patient monitoring system comprising:
a data acquisition device that can be coupled to a patient to acquire clinical data;
a patient monitor coupled to the medical acquisition device;
clinical data code displayed on the patient monitor, the clinical data code representing at least a portion of the acquired clinical data; and
a code reader that can be used by a caregiver to read the clinical data code in order to obtain at least a portion of the acquired clinical data from the patient monitor.
